(19) United States
(12) Patent Application Publication
(10) Pub. No.: US 2011/0145993 A1
(43) Pub. Date: Jun. 23, 2011

(54) INTEGRAL FLUID DISPOSAL APPARATUS AND SYSTEM

(75) Inventors: SHARON F. RADER, Pensacola, FL (US); Kara L. Rader-Phothimat, Holmdel, NJ (US); Matthew R. Rader, Middletown, CT (US); Regena L. Rader, Elkridge, MD (US)

(73) Assignee: Sharon F. Rader, Pensacola, FL (US)

(21) Appl. No.: 12/640,044

(22) Filed: Dec. 17, 2009

(51) Int. Cl. A61G 7/02; A61F 13/15

(52) U.S. Cl. 5/604; 604/378

(57) ABSTRACT

Embodiments of the present disclosure generally relate to a fluid disposal apparatus and system. In accordance with one embodiment of the present disclosure, a fluid disposal apparatus comprises a liquid collection device, a perforated support layer disposed above the liquid collection device, a spacer layer disposed above the perforated support layer comprising a plurality of substantially nonabsorbent members, and a perforated comfort layer disposed above the spacer layer.

### BACKGROUND

1. Field of the Disclosure

Embodiments of the present disclosure generally relate to an integral fluid disposal apparatus and system. More specifically, embodiments of the present disclosure relate to a fluid disposal apparatus and system, comprising at least one perforated or porous layer to allow fluid (e.g., urine) to be removed and collected in a liquid collection device to maintain the health and comfort of an incontinent sleeper.

2. Description of the Related Art

Urinary incontinence is a disorder that currently affects some 12 million Americans—a number which is steadily increasing. This condition, which can be caused and affected by a number of factors (e.g., age, gender, weight, stress), may prevent an individual from participating in social activities, going out in public, or otherwise carrying on a relatively normal life. In situations where the incontinence cannot be reversed, an affected individual may be forced to make arrangements, and sometimes severe lifestyle changes, to adapt to the condition.

One particular problem faced by individuals affected by urinary incontinence is finding comfort in sleeping. Occurrences of incontinence episodes during sleeping can range from occasional to frequent, depending on the severity of the condition, but are always uncomfortable and potentially embarrassing. Such episodes deter those affected by urinary incontinence from traveling overnight, planning long vacations, and other such activities. Additionally, incontinence episodes coupled with insufficient fluid removal may contribute to painful health conditions, such as urinary tract infections. For this reason, nursing homes are often evaluated for quality based in part on the number of urinary tract infection cases.

Previous attempts to provide assistance and comfort during sleep to individuals suffering from urinary incontinence have been wholly unsuccessful. Aside from providing adult diapers, which are known to cause self-confidence issues, discomfort issues, urinary tract infections, and the like, while adding to the overabundant waste problem occurring at landfills around the country, such attempts have included furnishing beds with an abundance of tanks, tubes, and vacuum pumps, as well as providing small apertures with access to a collection facility. However, not only are these previous attempts impractical (e.g., requiring a user, while asleep, to direct leakage through a small aperture in the middle of a bed), but they also cause more embarrassment than they seek to prevent (e.g., loud vacuum pumps expelling or isolating leaked fluids after each episode). As such, there has not yet been a feasible and effective solution to this problem.

Thus, there is a need for an integral fluid disposal system.

### SUMMARY

Embodiments of the present disclosure relate to a fluid disposal system, disposed, for example, in or on top of a bed, the fluid disposal system comprising multiple perforated or porous layers to allow fluid (e.g., urine) to be collected in a liquid collection device to maintain the comfort and sanitation of an incontinent sleeper. In accordance with one embodiment of the present disclosure, a fluid disposal apparatus comprises a liquid collection device, a perforated support layer disposed above the liquid collection device, a spacer layer disposed above the perforated support layer comprising a plurality of substantially nonabsorbent members, and a perforated comfort layer disposed above the spacer layer.

In accordance with another embodiment of the present disclosure, a fluid disposal apparatus comprises: a liquid collection device; a substantially rigid perforated support layer disposed above the liquid collection device, having an open weave structure; a spacer layer disposed above the support layer, comprising a plurality of substantially rigid, substantially nonabsorbent members, each member comprising an amount of a substantially firm and substantially flexible cushion disposed thereupon; and a perforated comfort layer disposed above the spacer layer and comprising an amount of a substantially firm and substantially flexible cushion disposed on at least a portion thereof, wherein the substantially flexible cushion may be a flexible gel.

In accordance with yet another embodiment of the present disclosure, a bed system comprises: a supporting frame and a fluid disposal apparatus, comprising: a liquid collection device; a perforated support layer disposed above the liquid collection device; a spacer layer comprising a plurality of waterproof members and disposed above the perforated support layer; and at least one perforated comfort layer disposed above the spacer layer.

### BRIEF DESCRIPTION OF THE DRAWINGS

So the manner in which the above recited features of the present disclosure can be understood in detail, a more particular description of embodiments of the present disclosure, briefly summarized above, may be had by reference to embodiments, which are illustrated in the appended drawings. It is to be noted, however, the appended drawings illustrate only typical embodiments of embodiments encompassed within the scope of the present disclosure, and, therefore, are not to be considered limiting, for the present disclosure may admit to other equally effective embodiments, wherein:

FIG. 1 depicts an exploded view of a fluid disposal apparatus in accordance with one embodiment of the present disclosure;

FIG. 2 depicts a perspective view of a spacer layer of a fluid disposal apparatus in accordance with another embodiment of the present disclosure;

FIG. 3 depicts an exploded view of a fluid disposal apparatus in accordance with yet another embodiment of the present disclosure;

FIG. 4 depicts an exploded view of a fluid disposal apparatus in accordance with yet another embodiment of the present disclosure;

FIG. 5 depicts a cross-sectional view of a fluid disposal apparatus in accordance with yet another embodiment of the present disclosure;

FIG. 6 depicts a fluid disposal system with an integral fluid disposal apparatus in accordance with yet another embodiment of the present disclosure;

FIG. 7 depicts an exploded view of a fluid disposal apparatus in accordance with yet another embodiment of the present disclosure;

FIG. 8 depicts a top view of a fluid disposal apparatus in accordance with yet another embodiment of the present disclosure;

FIG. 9 depicts a cross-sectional view of a fluid disposal apparatus in accordance with yet another embodiment of the present disclosure;

FIG. 10 depicts a perspective front view of a cloak apparatus in accordance with yet another embodiment of the present disclosure;

FIG. 11 depicts a perspective top view of a cloak inner layer in accordance with yet another embodiment of the present disclosure; and

FIG. 12 depicts a perspective front view of a cloak apparatus in accordance with yet another embodiment of the present disclosure.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

### DETAILED DESCRIPTION

In the following detailed description, numerous specific details are set forth in order to provide a thorough understanding of exemplary embodiments or other examples described herein. However, it will be understood these examples may be practiced without the specific details. In other instances, well-known methods, procedures, and components have not been described in detail, so as to not obscure the following description. Furthermore, the examples disclosed herein are for exemplary purposes only and other examples may be employed in lieu of, or in combination with, the examples disclosed.

Embodiments of the present disclosure generally relate to an integral fluid disposal apparatus, which can be disposed as a system, for example, in a bed, a bed insert, or a bed topper. More specifically, embodiments of the present disclosure relate to a fluid disposal apparatus, comprising at least one perforated or porous layer to allow fluid (e.g., urine) to be collected in a liquid collection device to maintain the health and comfort of an incontinent sleeper.

As used herein, the term “perforated” and any derivative term thereof may refer to the condition of containing holes, perforations, or interstices disposed therein, either as an added characteristic (e.g., the introduction of holes into the material) or as a natural characteristic of the material (e.g., open weave construction of some fabric materials).

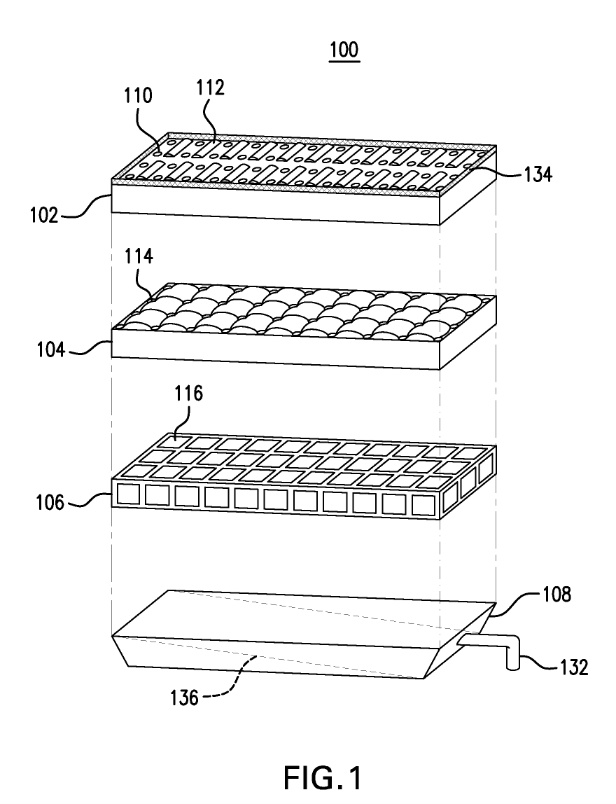

Referring now to FIG. 1, a fluid disposal apparatus 100 is depicted in accordance with one embodiment of the present disclosure. The fluid disposal apparatus 100 may comprise at least a liquid collection device 108, a support layer 106, a spacer layer 104, and a comfort layer 102. Although the layers of the fluid disposal apparatus are depicted in FIG. 1 in a specific configuration (i.e., from top to bottom: comfort layer 102, spacer layer 104, support layer 106, liquid collection device 108), it should be noted that any configuration may be implemented without departing from the scope of embodiments of the present disclosure.

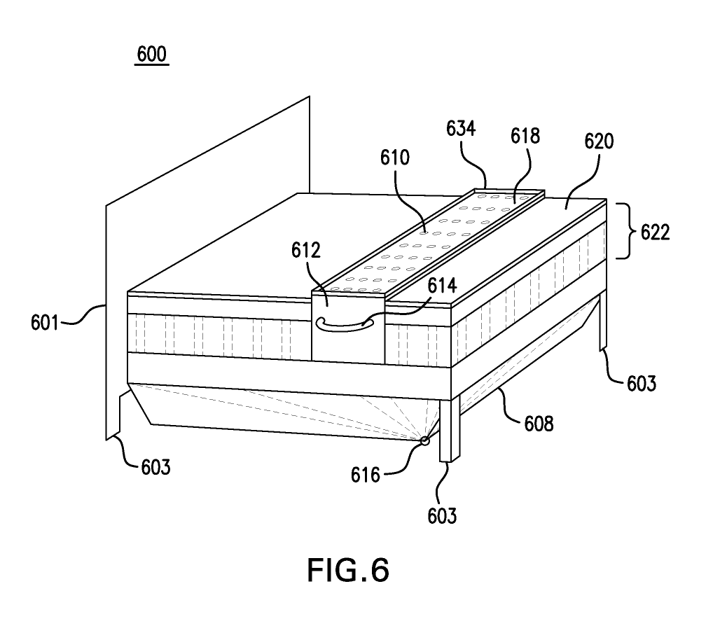

The comfort layer 102 may be configured as the top portion of the fluid disposal apparatus 100, as depicted in FIG. 1. The comfort layer 102 may be provided for the comfort of the user, as the user may sleep directly on the comfort layer 102 or on a top cover (see FIG. 9). A collar 134 for attaching a fabric device cover (see FIG. 6) or a top cover (see FIG. 9) may comprise a lip and padding for the comfort of the user. The collar 134 may be designed in the shape of, and function like, an embroidery ring or any other shape. The collar 134 may comprise, for example, an outer frame and an inner frame (not pictured), wherein the outer frame fits on top of and substantially covers the outside surface of the inner frame and is secured frictionally to the inner frame. Although the collar 134 is depicted on top of the comfort layer 102, the collar 134 may be attached to the top of any layer, including, but not limited to the spacer layer 104, the support layer 106, and the liquid collection device 108.

In accordance with one embodiment of the present disclosure, the comfort layer 102 may comprise a flexible material that provides sufficient conformability to cushion a user's body, such as an open-celled polymer foam, a flexible gel, or the like or may comprise a case filled with feathers, down, synthetic fill, and the like, to achieve the same end. The comfort layer 102 may also comprise a substantially nonabsorbent material, such as a polymeric material, which would allow a leaked fluid, such as urine, to be easily transported and removed from at least a portion of the fluid disposal apparatus 100.

Additionally, the comfort layer 102 may be perforated. As depicted in FIG. 1, all or any portion of the comfort layer 102 may comprise one or more holes 110 or interstitial spaces through which a fluid may pass. The holes 110 may take any dimensions (e.g., size, shape) feasible in embodiments of the present disclosure. It should also be noted that each of the holes 110 may be identical, substantially similar, varied or substantially different in any one or more dimensions or characteristics. The comfort layer 102 may comprise a height gradient, having a maximum height at the areas furthest away from the holes 110 and a minimum height at the region or regions of the comfort layer 102 containing the holes 110. The shape of the comfort layer 102 may facilitate the flow of leaked fluids through the holes 110. The comfort layer 102 may include a flexible gel 112 for cushioning.

Disposed below the comfort layer 102 may be a spacer layer 104. The spacer layer 104 may serve to separate the comfort layer 102, which may come in contact with the user, from the lower two layers—namely, the support layer 106 and the liquid collection device 108. The spacer layer 104 may additionally facilitate the flow of leaked fluid through the fluid disposal apparatus 100. The spacer layer 106 may include a flexible gel for cushioning.

As depicted in FIG. 1, the spacer layer 104 may comprise at least one substantially nonabsorbent member 114 in accordance with one embodiment of the present disclosure. The at least one member 114 creates or provides a channel or void space between the comfort layer 102 and the support layer 106 such that leaked fluid is allowed to transition between layers in a relatively channeled fashion. Although the at least one member 114 of the spacer layer 104 are depicted in FIG. 1 as cylindrical or setaceous bodies, the at least one member 114 may comprise any dimensions (e.g., shape, size) feasible in embodiments of the present disclosure and may be topped with a flexible silicone gel for added flexibility. The at least one member 114 may additionally comprise a substantially nonabsorbent material, such as a polymeric material. This choice of material would allow leaked fluids to flow between the at least one member 114 and through the spacer layer 104. In such a manner, the fluid is capable of reaching its final destination (e.g., the liquid collecting device 108 or otherwise) with minimal difficulty and interference (e.g., caused by absorption of leaked fluid). The spacer layer 104 may comprise a height gradient, having a maximum height at the areas of the spacer layer 104 which are furthest away from the at least one member 114 and a minimum height at the region or regions of the spacer layer 104 containing the at least one member 114. The shape of the spacer layer 104 may facilitate the escape of leaked fluids through the at least one member 114.

A support layer 106 may be configured below the spacer layer 104. The support layer 106 may provide support and/or stability to the spacer layer 104 and the comfort layer 102, as well as providing a second degree of separation between the upper layers and the liquid collection device 108. The support layer 106 may comprise any substantially rigid or semi-rigid material, including polymeric materials. The material and structure of the support layer 106 should be sufficient to support the weight of at least one person.

As depicted in FIG. 1, the support layer 106 may additionally be perforated, as discussed above with reference to the comfort layer 102. In such an embodiment, at least a portion of the support layer 106 may comprise one or more holes 116. The presence of the holes 116 in the support layer 106 allows any leaked fluid to continue its pathway down through the system and into the liquid collection device 108 or other final destination. Although depicted in FIG. 1 as holes covering the majority of the support layer 106, it should be noted that the holes 116 may take any dimensions (e.g., shape, size) and may cover all or any portion of the support layer 106 feasible in embodiments of the present disclosure without departing from the scope of embodiments of the present disclosure.

The support layer 106 may comprise a height gradient, having a maximum height at the areas of the support layer 106 which are furthest away from the holes 116 and a minimum height at the region or regions of the support layer 106 containing the holes 116. The shape of the support layer 106 may facilitate the escape of leaked fluids through the holes 116.

The liquid collection device 108 may be disposed at the bottom of the fluid disposal apparatus 100, as depicted in FIG. 1. The liquid collection device 108 may comprise any device sufficient to capture and contain fluid, including but not limited to one or more tanks, troughs, containers, and the like. The liquid collection device 108 may optionally be covered on a portion thereof, for example, for the purpose of minimizing the escape of odors and fluid.

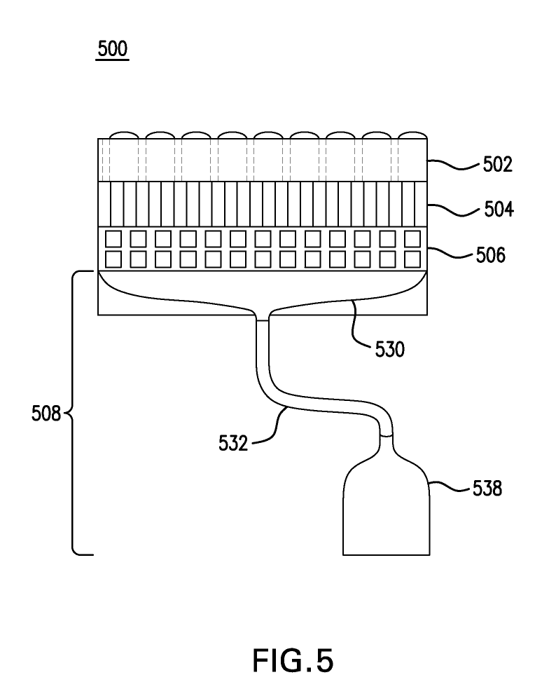

The liquid collection device 108 may comprise any material that would serve to prevent collected fluid from escaping into the surroundings of the fluid disposal system 100. This material may include any substantially nonabsorbent material or plurality of materials feasible for embodiments of the present disclosure, including but not limited to polymeric materials, metals, other such materials, and blends and alloys thereof. In accordance with another embodiment of the present disclosure, the liquid collection device 108 may comprise an absorbent or superabsorbent material, such as open-cell cellulose, which may allow the liquid collection device 108 to retain the fluid within the material. In such an embodiment, the fluid may be removed from the liquid collection device 108 via drying, vacuuming, evaporation, rinsing, washing, and the like, and reused in further applications. A funnel (see FIG. 5) may be disposed on top of the liquid collection device 108. An angled chamber 136 may be contained within the liquid collection device 108 for assisting in draining the liquid. A conduit 132 may be disposed on the side of the liquid collection device 108 for draining the liquid.

With reference to FIG. 1, in accordance with another embodiment of the present disclosure, the fluid disposal apparatus 100 may be disposed in a bed (see FIG. 6) and removable. The fluid disposal apparatus 100 may be removed, for example, using a drawer track and rail system, draw string, velcro, hooks, snaps, buttons, and the like.

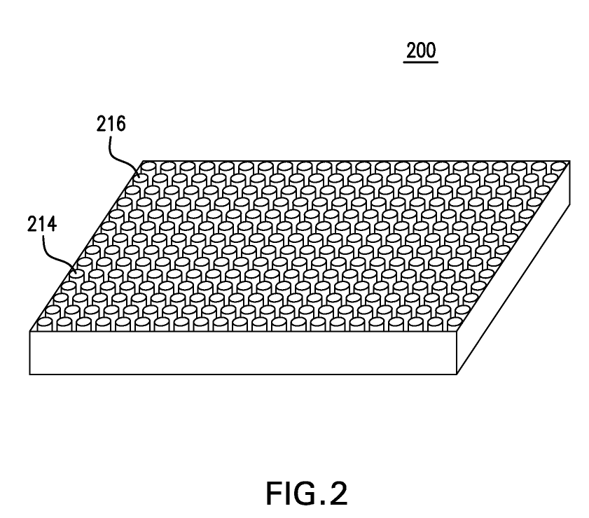

With reference to FIG. 2, in accordance with another embodiment of the present disclosure, a cushion 216 may be disposed on at least one substantially nonabsorbent member 214 of a spacer layer 200 of a fluid disposal apparatus. The cushion 216 may serve to provide the user with a softer or more conformable and flexible sleeping surface. Although depicted in FIG. 2 as being disposed at least one substantially nonabsorbent member 214 of a spacer layer 200, it should be noted that a cushion 216 may be disposed on any one or more members 214 of a spacer layer 200, or on any portion or all of any component of a fluid disposal apparatus 100 in FIG. 1 (e.g., comfort layer 102, spacer layer 104, support layer 106) without departing from the scope of embodiments of the present disclosure. In accordance with another embodiment of the disclosure, at least one substantially nonabsorbent member 214 may be a rubber finger-like projection.

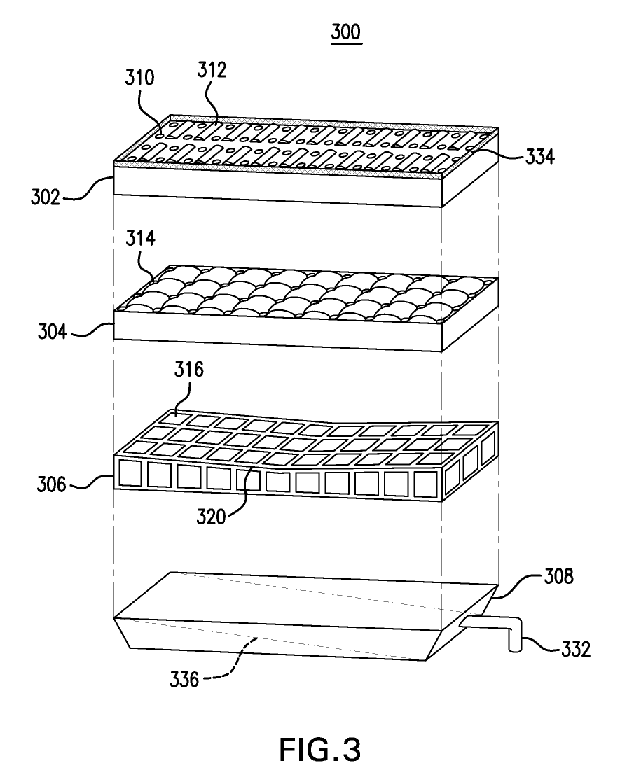

Referring now to FIG. 3, a fluid disposal apparatus 300 is depicted in accordance with one embodiment of the present disclosure. The fluid disposal apparatus 300 may comprise at least a liquid collection device 308, a support layer 306, a spacer layer 304, and a comfort layer 302. Although the layers of the fluid disposal apparatus are depicted in FIG. 3 in a specific configuration (i.e., from top to bottom: comfort layer 302, spacer layer 304, support layer 306, liquid collection device 308), it should be noted that any configuration may be implemented without departing from the scope of embodiments of the present disclosure.

The fluid disposal apparatus 300 may further comprise a substantially V-shaped or concave drainage channel 320. The drainage channel 320 may comprise an indentation or depression in at least a portion of the layer on which it is disposed. The layer in which the drainage channel 320 is disposed may additionally comprise substantially slanted or sloped portions that may serve to facilitate the flow of fluid into the drainage channel 320. It should be noted that, although the drainage channel 320 is depicted as being disposed on the support layer 306, the drainage channel 320 may be disposed on any one or more of the comfort layer 302, the spacer layer 304, or the support layer 306.

The comfort layer 302 may be configured as the top portion of the fluid disposal apparatus 300, as depicted in FIG. 3. The comfort layer 302 may be provided for the comfort of the user, as the user may sleep directly on the comfort layer 302 or on a top cover (see FIG. 9). A collar 334 for attaching a fabric device cover (see FIG. 6) or a top cover (see FIG. 9) may comprise a lip and padding for the comfort of the user. The fabric device cover (see FIG. 6) and the top cover (see FIG. 9), may be detachably attached to the collar 134 with a draw string. The collar 334 may be designed in the shape of, and function like, an embroidery ring or any other shape. The collar 334 may comprise, for example, an outer frame and an inner frame (not pictured), wherein the outer frame fits on top of and substantially covers the outside surface of the inner frame and is secured frictionally to the inner frame. Although the collar 334 is depicted on top of the comfort layer 302, the collar 334 may be attached to the top of any layer, including, but not limited to the spacer layer 304, the support layer 306, and the liquid collection device 308.

In accordance with one embodiment of the present disclosure, the comfort layer 302 may comprise a flexible material that provides sufficient conformability to cushion a user's body, such as an open-celled polymer foam, a flexible gel, or the like or may comprise a case filled with feathers, down, synthetic fill, and the like, to achieve the same end. The comfort layer 302 may also comprise a substantially nonabsorbent material, such as a polymeric material, which would allow a leaked fluid, such as urine, to be easily transported and removed from at least a portion of the fluid disposal apparatus 300.

Additionally, the comfort layer 302 may be perforated. As depicted in FIG. 3, all or any portion of the comfort layer 302 may comprise one or more holes 310 or interstitial spaces through which a fluid may pass. The holes 310 may take any dimensions (e.g., size, shape) feasible in embodiments of the present disclosure. It should also be noted that each of the holes 310 may be identical, substantially similar, varied or substantially different in any one or more dimensions or characteristics. The comfort layer 302 may comprise a height gradient, having a maximum height at the areas furthest away from the holes 310 and a minimum height at the region or regions of the comfort layer 302 containing the holes 310. The shape of the comfort layer 302 may facilitate the flow of leaked fluids through the holes 310. The comfort layer 302 may include a flexible gel 312 for cushioning.

Disposed below the comfort layer 302 may be a spacer layer 304. The spacer layer 304 may serve to separate the comfort layer 302, which may come in contact with the user, from the lower two layers—namely, the support layer 306 and the liquid collection device 308. The spacer layer 304 may additionally facilitate the flow of leaked fluid through the fluid disposal apparatus 300. The spacer layer 306 may include a flexible gel for cushioning.

As depicted in FIG. 3, the spacer layer 304 may comprise at least one substantially nonabsorbent member 314 in accordance with one embodiment of the present disclosure. The at least one member 314 creates or provides a channel or void space between the comfort layer 302 and the support layer 306 such that leaked fluid is allowed to transition between layers in a relatively channeled fashion. Although the at least one member 314 of the spacer layer 304 are depicted in FIG. 3 as cylindrical or setaceous bodies, the at least one member 314 may comprise any dimensions (e.g., shape, size) feasible in embodiments of the present disclosure and may be topped with a flexible silicone gel for added flexibility. The at least one member 314 may additionally comprise a substantially nonabsorbent material, such as a polymeric material. This choice of material would allow leaked fluids to flow between the at least one member 314 and through the spacer layer 304. In such a manner, the fluid is capable of reaching its final destination (e.g., the liquid collecting device 308 or otherwise) with minimal difficulty and interference (e.g., caused by absorption of leaked fluid). The spacer layer 304 may comprise a height gradient, having a maximum height at the areas of the spacer layer 304 which are furthest away from the at least one member 314 and a minimum height at the region or regions of the spacer layer 304 containing the at least one member 314. The shape of the spacer layer 304 may facilitate the escape of leaked fluids through the at least one member 314.

A support layer 306 may be configured below the spacer layer 304. The support layer 306 may provide support and/or stability to the spacer layer 304 and the comfort layer 302, as well as providing a second degree of separation between the upper layers and the liquid collection device 308. The support layer 306 may comprise any substantially rigid or semi-rigid material, including polymeric materials. The material and structure of the support layer 306 should be sufficient to support the weight of at least one person.

As depicted in FIG. 3, the support layer 306 may additionally be perforated, as discussed above with reference to the comfort layer 302. In such an embodiment, at least a portion of the support layer 306 may comprise one or more holes 316. The presence of the holes 316 in the support layer 306 allows any leaked fluid to continue its pathway down through the system and into the liquid collection device 308 or other final destination. Although depicted in FIG. 3 as holes covering the majority of the support layer 306, it should be noted that the holes 316 may take any dimensions (e.g., shape, size) and may cover all or any portion of the support layer 306 feasible in embodiments of the present disclosure without departing from the scope of embodiments of the present disclosure.

The support layer 306 may comprise a height gradient, having a maximum height at the areas of the support layer 306 which are furthest away from the holes 316 and a minimum height at the region or regions of the support layer 306 containing the holes 316. The shape of the support layer 306 may facilitate the escape of leaked fluids through the holes 316.

The liquid collection device 308 may be disposed at the bottom of the fluid disposal apparatus 300, as depicted in FIG. 3. The liquid collection device 308 may comprise any device sufficient to capture and contain fluid, including but not limited to one or more tanks, troughs, containers, and the like. The liquid collection device 308 may optionally be covered on a portion thereof, for example, for the purpose of minimizing the escape of odors and fluid.

The liquid collection device 308 may comprise any material that would serve to prevent collected fluid from escaping into the surroundings of the fluid disposal system 300. This material may include any substantially nonabsorbent material or plurality of materials feasible for embodiments of the present disclosure, including but not limited to polymeric materials, metals, other such materials, and blends and alloys thereof. In accordance with another embodiment of the present disclosure, the liquid collection device 308 may comprise an absorbent or superabsorbent material, such as open-cell cellulose, which may allow the liquid collection device 308 to retain the fluid within the material. In such an embodiment, the fluid may be removed from the liquid collection device 308 via drying, vacuuming, evaporation, rinsing, washing, and the like, and reused in further applications. A funnel (see FIG. 5) may be disposed on top of the liquid collection device 308. An angled chamber 316 may be contained within the liquid collection device 308 for assisting in draining the liquid. A conduit 312 may be disposed on the side of the liquid collection device 308 for draining the liquid.

With reference to FIG. 3, in accordance with another embodiment of the present disclosure, the fluid disposal apparatus 300 may be disposed in a bed (see FIG. 6) and removable. The fluid disposal apparatus 300 may be removed, for example, using a drawer track and rail system, draw string, velcro, hooks, snaps, buttons, and the like.

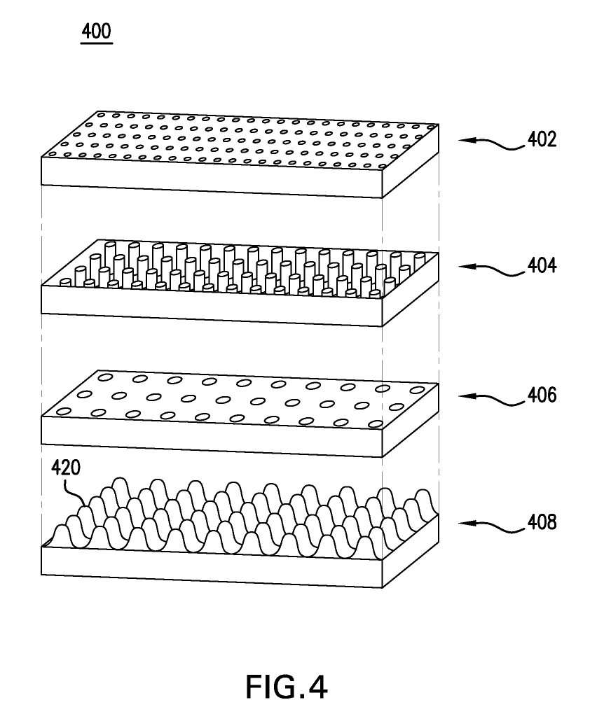

Referring now to FIG. 4, the liquid collection device 408 of a fluid disposal apparatus 400 may comprise one or more cuplike recesses 420 disposed in at least a portion of the liquid collection device 408. Each of the recesses 420 may comprise a depression or indentation adapted to contain an amount of leaked liquid. In accordance with one embodiment of the present disclosure, the liquid collection device 408 may take a shape generally substantially similar to that of any one or more of the comfort layer 402, the spacer layer 404, and the support layer 406. In such an embodiment, the recesses 420 may be concealed beneath one or more other layers, thus contributing to, and enhancing, the aesthetic appearance of the fluid disposal apparatus 400, particularly with respect to the liquid collection device 408.

Referring now to FIG. 5, another embodiment of the present disclosure is depicted in which the liquid collection device 508 of the fluid disposal apparatus 500 comprises at least a funnel 530, a conduit 532, and a liquid storage container 538. In such an embodiment, the funnel 530 may be positioned beneath each of a comfort layer 502, a spacer layer 504, and a support layer 506, and adapted to capture fluid falling from any of the aforementioned layers. As depicted, the funnel 530, which may be disposed beneath all or a portion of the bottommost layer (in FIG. 5, the support layer 506), may be attached at the stem to a conduit 532. The conduit 532 may comprise any tubing or transport mechanism feasible in embodiments of the present disclosure. Another end of the conduit 532 may be attached to, disposed in, or disposed on an open end of a liquid storage container 538. The liquid storage container 538 may comprise a bottle, bag, cylinder, measuring device, or any other container feasible in embodiments of the present disclosure.

It should be noted that, although depicted as generally rectangular in shape, any portions of a fluid disposal apparatus in accordance with one or more embodiments of the present disclosure, including but not limited to comfort layers, spacer layers, support layers, and liquid collection devices, may take any size, shape, or dimensions without departing from the scope of embodiments of the present disclosure. Each of the portions of a fluid disposal apparatus may be identical, substantially similar, variable or substantially different in any one or more dimensions.

Referring to FIG. 5, in accordance with one embodiment of the present disclosure, the fluid disposal apparatus 500 may be washable and/or reusable. This may be achieved by forming at least the majority of the components of the fluid disposal apparatus from substantially nonabsorbent materials, such as polymeric materials (e.g., polyethylene, polypropylene, polyethylene terephthalate, butadiene rubber, etc.), metals or metal alloys (e.g., aluminum, iron, steel, stainless steel, brass, etc.), or any other such material of construction feasible in embodiments of the present disclosure. In such a manner, the fluid disposal apparatus may be washed, cleaned, and reused, preventing unnecessary expenses and refuse and providing an environmentally-friendly product.

Referring now to FIG. 6, in accordance with one embodiment of the present disclosure, a fluid disposal system 600 may comprise a supporting frame 601, a plurality of legs 603, and a fluid disposal apparatus 612 comprising a comfort layer (see FIG. 1), a spacer layer (see FIG. 1), a support layer (see FIG. 1), a padding layer 622, a fabric device cover 620 and a liquid collection device 608. The supporting frame 601 may comprise any one or more of a headboard, a footboard, a set of rails connecting the headboard and the footboard, or any other components of a traditional or unconventional bed frame without departing from the scope of embodiments of the present disclosure. The plurality of legs 603 provides support and stability to at least a portion of the bed frame 601. It should also be noted that any of the components of the fluid disposal apparatus may comprise any of the properties, accessories, materials, or any other characteristics described above with reference to other embodiments of the fluid disposal apparatus. A top cover 618 may cover at least a portion of the fluid disposal apparatus 612 and may be detachably secured to a collar 634. The top cover 618 may be of any size or shape suitable for covering the fluid disposal apparatus 612 and accommodating a person of any size and weight. The top cover 618 may comprise any material that would be suitable for accommodating a person of any size and weight and having any level of firmness to accommodate any comfort preference.

A padding layer 622 positioned on the frame 601 disposed adjacent to the fluid disposal apparatus 612 may comprise any material suitable for cushioning such as such as open-celled polymer foam, flexible gel, mattress material, feathers, down, synthetic fill, and the like, to achieve the same end. In accordance with another embodiment of the present disclosure, a fabric device cover 620 may surround the fluid disposal apparatus 612. In accordance with one embodiment of the present disclosure, the fabric device cover 620 may comprise one or more padded sheets, removable and washable absorbent fabric pads, removable and disposable absorbent fabric pads, foam pads, blankets, rubber mats, disposable cotton cushions, or the like, separating the user from the surface of padding layer 622. The fabric device cover 620 may be detachably attached to a collar 634 affixed to the top of the padding layer 622. The fabric device cover 620 may be provided for the comfort of the user. The collar 634 may comprise a lip and padding for the comfort of the user. The collar 634 may be designed in the shape of, and function like, an embroidery ring or any other shape. The collar 634 may comprise, for example, an outer frame and an inner frame (not pictured), wherein the outer frame fits on top of and substantially covers the outside surface of the inner frame and is secured frictionally to the inner frame. Although the collar 634 is depicted on top of the padding layer 622, the collar 634 may be attached to the top of any layer.

In operation, a user may lay on the padding layer 622, either directly (e.g., uncovered) or indirectly (e.g., wherein at least the comfort layer (see FIG. 1) is covered at least partially by the top cover 618). If the user should emit any sort of fluid (e.g., during an episode of urinary incontinence), the leaked fluid would fall through the holes 610 disposed in the comfort layer (see FIG. 1). From there, the leaked fluid may flow down through the spacer layer (see FIG. 1) and onto the support layer (see FIG. 1). As the support layer (see FIG. 1) is perforated, the leaked fluid may flow down through the holes in the support layer (see FIG. 1), into the liquid collection device 608. The leaked fluid is captured in the liquid collection device 608, which may then be emptied and potentially cleaned. The fluid disposal system 600 may comprise a drainage aperture 616 disposed below the liquid collection device 608 for emptying collected liquid. The fluid disposal apparatus 612 may be removable for ease of cleaning. The fabric device cover 620 may contain an aperture or cut-out sized to an opening of the fluid disposal apparatus 612. Draw string, velcro, snaps, and the like may be used to secure the removable fabric device cover 620. A handle 614 may be affixed to the removable portion 612 for removing the portion from the system.

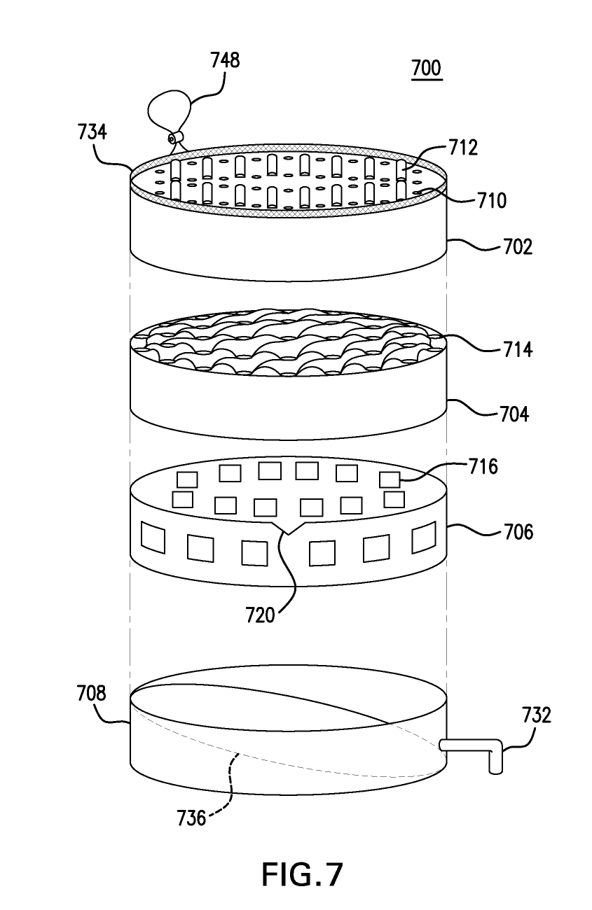

Referring now to FIG. 7, a fluid disposal apparatus 700 is depicted in accordance with another embodiment of the present disclosure. The fluid disposal apparatus 700 may comprise at least a liquid collection device 708, a support layer 706, a spacer layer 704, and a comfort layer 702. Although the layers of the fluid disposal apparatus are depicted in FIG. 7 in a specific configuration (i.e., from top to bottom: comfort layer 702, spacer layer 704, support layer 706, liquid collection device 708), it should be noted that any configuration may be implemented without departing from the scope of embodiments of the present disclosure.

The fluid disposal apparatus 700 may further comprise a substantially concave drainage channel 720. The drainage channel 720 may comprise an indentation or depression in at least a portion of the layer on which it is disposed. The layer in which the drainage channel 720 is disposed may additionally comprise substantially slanted or sloped portions that may serve to facilitate the flow of fluid into the drainage channel 720. It should be noted that, although the drainage channel 720 is depicted as being disposed on the support layer 706, the drainage channel 720 may be disposed on any one or more of the comfort layer 702, the spacer layer 704, or the support layer 706.

The comfort layer 702 may be configured as the top portion of the fluid disposal apparatus 700, as depicted in FIG. 7. The comfort layer 702 may be provided for the comfort of the user, as the user may sleep directly on the comfort layer 702 or on a top cover (see FIG. 9). A collar 734 for attaching a fabric device cover (see FIG. 6) or a top cover (see FIG. 9) may comprise a lip and padding for the comfort of the user. The fabric device cover (see FIG. 6) and the top cover (see FIG. 9) may be detachably attached to the collar 734 with a draw string 748 disposed substantially within the collar 734 and extending at least the perimeter of the collar 734. The collar 734 may be designed in the shape of, and function like, an embroidery ring or any other shape. The collar 734 may comprise, for example, an outer frame and an inner frame (not pictured), wherein the outer frame fits on top of and substantially covers the outside surface of the inner frame and is secured frictionally to the inner frame. Although the collar 734 is depicted on top of the comfort layer 702, the collar 734 may be attached to the top of any layer, including, but not limited to the spacer layer 704, the support layer 706, and the liquid collection device 708.

In accordance with one embodiment of the present disclosure, the comfort layer 702 may comprise a flexible material that provides sufficient conformability to cushion a user's body, such as an open-celled polymer foam, a flexible gel, or the like or may comprise a case filled with feathers, down, synthetic fill, and the like, to achieve the same end. The comfort layer 702 may also comprise a substantially nonabsorbent material, such as a polymeric material, which would allow a leaked fluid, such as urine, to be easily transported and removed from at least a portion of the fluid disposal apparatus 700.

Additionally, the comfort layer 702 may be perforated. As depicted in FIG. 7, all or any portion of the comfort layer 702 may comprise one or more holes 710 or interstitial spaces through which a fluid may pass. The holes 710 may take any dimensions (e.g., size, shape) feasible in embodiments of the present disclosure. It should also be noted that each of the holes 710 may be identical, substantially similar, varied or substantially different in any one or more dimensions or characteristics. The comfort layer 702 may comprise a height gradient, having a maximum height at the areas furthest away from the holes 710 and a minimum height at the region or regions of the comfort layer 702 containing the holes 710. The shape of the comfort layer 702 may facilitate the flow of leaked fluids through the holes 710. The comfort layer 702 may include a flexible gel 712 for cushioning.

Disposed below the comfort layer 702 may be a spacer layer 704. The spacer layer 704 may serve to separate the comfort layer 702, which may come in contact with the user, from the lower two layers—namely, the support layer 706 and the liquid collection device 708. The spacer layer 704 may additionally facilitate the flow of leaked fluid through the fluid disposal apparatus 700. The spacer layer 706 may include a flexible gel for cushioning.

As depicted in FIG. 7, the spacer layer 704 may comprise at least one substantially nonabsorbent member 714 in accordance with one embodiment of the present disclosure. The at least one member 714 creates or provides a channel or void space between the comfort layer 702 and the support layer 706 such that leaked fluid is allowed to transition between layers in a relatively channeled fashion. Although the at least one member 714 of the spacer layer 704 are depicted in FIG. 7 as cylindrical or setaceous bodies, the at least one member 714 may comprise any dimensions (e.g., shape, size) feasible in embodiments of the present disclosure and may be topped with a flexible silicone gel for added flexibility. The at least one member 714 may additionally comprise a substantially nonabsorbent material, such as a polymeric material. This choice of material would allow leaked fluids to flow between the at least one member 714 and through the spacer layer 704. In such a manner, the fluid is capable of reaching its final destination (e.g., the liquid collecting device 708 or otherwise) with minimal difficulty and interference (e.g., caused by absorption of leaked fluid). The spacer layer 704 may comprise a height gradient, having a maximum height at the areas of the spacer layer 704 which are furthest away from the at least one member 714 and a minimum height at the region or regions of the spacer layer 704 containing the at least one member 714. The shape of the spacer layer 704 may facilitate the escape of leaked fluids through the at least one member 714.

A support layer 706 may be configured below the spacer layer 704. The support layer 706 may provide support and/or stability to the spacer layer 704 and the comfort layer 702, as well as providing a second degree of separation between the upper layers and the liquid collection device 708. The support layer 706 may comprise any substantially rigid or semi-rigid material, including polymeric materials. The material and structure of the support layer 706 should be sufficient to support the weight of at least one person.

As depicted in FIG. 7, the support layer 706 may additionally be perforated, as discussed above with reference to the comfort layer 702. In such an embodiment, at least a portion of the support layer 706 may comprise one or more holes 716. The presence of the holes 716 in the support layer 706 allows any leaked fluid to continue its pathway down through the system and into the liquid collection device 708 or other final destination. Although depicted in FIG. 7 as holes covering the majority of the support layer 706, it should be noted that the holes 716 may take any dimensions (e.g., shape, size) and may cover all or any portion of the support layer 706 feasible in embodiments of the present disclosure without departing from the scope of embodiments of the present disclosure.

The support layer 706 may comprise a height gradient, having a maximum height at the areas of the support layer 706 which are furthest away from the holes 716 and a minimum height at the region or regions of the support layer 706 containing the holes 716. The shape of the support layer 706 may facilitate the escape of leaked fluids through the holes 716.

The liquid collection device 708 may be disposed at the bottom of the fluid disposal apparatus 700, as depicted in FIG. 7. The liquid collection device 708 may comprise any device sufficient to capture and contain fluid, including but not limited to one or more tanks, troughs, containers, and the like. The liquid collection device 708 may optionally be covered on a portion thereof, for example, for the purpose of minimizing the escape of odors and fluid.

The liquid collection device 708 may comprise any material that would serve to prevent collected fluid from escaping into the surroundings of the fluid disposal system 700. This material may include any substantially nonabsorbent material or plurality of materials feasible for embodiments of the present disclosure, including but not limited to polymeric materials, metals, other such materials, and blends and alloys thereof. In accordance with another embodiment of the present disclosure, the liquid collection device 708 may comprise an absorbent or superabsorbent material, such as open-cell cellulose, which may allow the liquid collection device 708 to retain the fluid within the material. In such an embodiment, the fluid may be removed from the liquid collection device 708 via drying, vacuuming, evaporation, rinsing, washing, and the like, and reused in further applications. A funnel (see FIG. 5) may be disposed on top of the liquid collection device 708. An angled chamber 736 may be contained within the liquid collection device 708 for assisting in draining the liquid. A conduit 732 may be disposed on the side of the liquid collection device 708 for draining the liquid.

With reference to FIG. 7, in accordance with another embodiment of the present disclosure, the fluid disposal apparatus 700 may be disposed in a bed (see FIG. 6) and removable. The fluid disposal apparatus 700 may be removed, for example, using a drawer track and rail system, draw string, velcro, hooks, snaps, buttons, and the like.

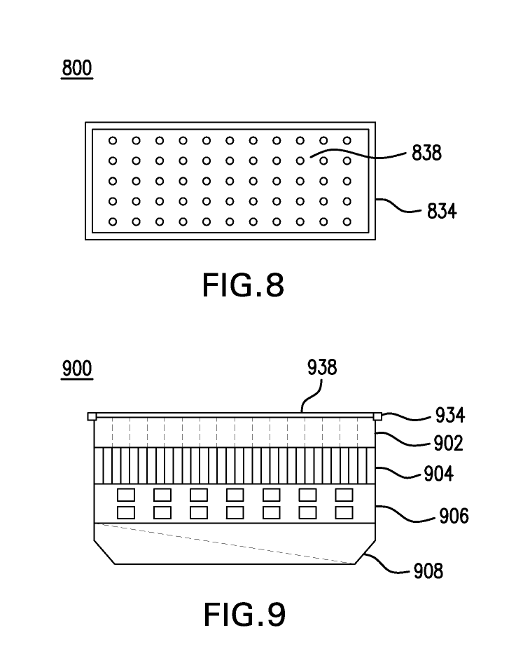

With reference to FIG. 8, in accordance with another embodiment of the present disclosure, the fluid disposal apparatus 800 may comprise a top cover 838 covering at least a portion of the fluid disposal apparatus 800. The top cover 838 may be detachably secured to a collar 834. The top cover 838 may be of any size or shape suitable for covering the fluid disposal apparatus 800 and accommodating a person of any size and weight. The top cover 838 may comprise material having any level of firmness to accommodate any comfort preference. The collar 834 may comprise a lip and padding for the comfort of the user. The collar 834 may be designed in the shape of, and function like, an embroidery ring or any other shape. The collar 834 may comprise, for example, an outer frame and an inner frame (not pictured), wherein the outer frame fits on top of and substantially covers the outside surface of the inner frame and is secured frictionally to the inner frame.

With reference to FIG. 9, in accordance with another embodiment of the present disclosure, a fluid disposal apparatus 900 may comprise at least a liquid collection device 908, a support layer 906, a spacer layer 904, and a comfort layer 902. Although the layers of the fluid disposal apparatus are depicted in FIG. 9 in a specific configuration (i.e., from top to bottom: comfort layer 902, spacer layer 904, support layer 906, liquid collection device 908), it should be noted that any configuration may be implemented without departing from the scope of embodiments of the present disclosure.

Referring to FIG. 9, in accordance with another embodiment of the present disclosure, the fluid disposal apparatus 900 may comprise a top cover 938 covering at least a portion of the fluid disposal apparatus 900. The top cover 938 may be detachably secured to a collar 934. The top cover 938 may be of any size or shape suitable for covering the fluid disposal apparatus 900 and accommodating a person of any size and weight. The top cover 938 may comprise a material having any level of firmness to accommodate any comfort preference. The collar 934 may comprise a lip and padding for the comfort of the user. The collar 934 may be designed in the shape of, and function like, an embroidery ring or any other shape. The collar 934 may comprise an outer frame and an inner frame (not pictured), wherein the outer frame fits on top of and substantially covers the outside surface of the inner frame and is secured frictionally to the inner frame. It should be noted that, although depicted as generally rectangular in shape, any portion of a fluid disposal apparatus 900 in accordance with one or more embodiments of the present disclosure, including but not limited to comfort layers, spacer layers, support layers, and liquid collection devices, may take any size, shape, or dimensions without departing from the scope of embodiments of the present disclosure. Each of the portions of the fluid disposal apparatus may be identical, substantially similar, variable or substantially different in any one or more dimensions.

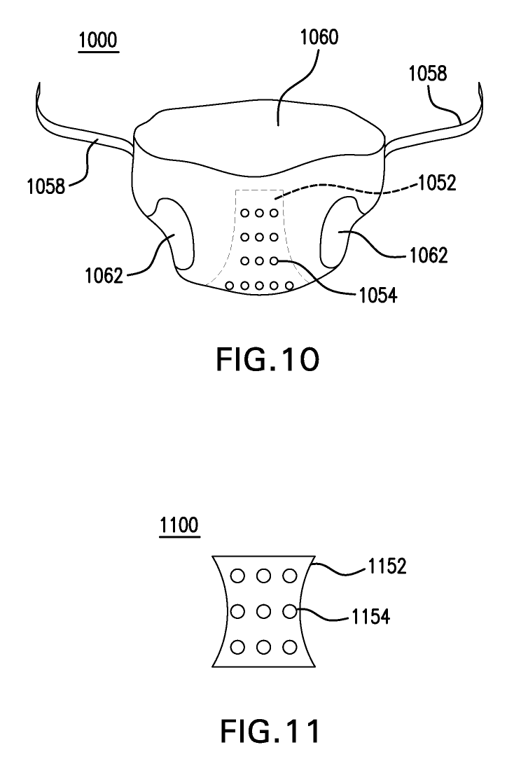

Referring to FIG. 10, there is provided a perspective front view of a cloak. In accordance with one embodiment of the present disclosure, a fluid disposal system comprises a cloak 1000 for providing user privacy and comfort, the cloak detachably attached to a collar (not pictured) secured to a fluid disposal apparatus (not pictured). The cloak 1000 may comprise a non-absorbent fabric 1052 having a plurality of drainage holes 1054 and a sufficient size and shape to accommodate a person of any size and weight. The cloak 1000 may further comprise a top aperture 1060 for accepting the waist of a person, two lower apertures 1062 for accepting the legs of the person, and a fastening device 1058 for securing the cloak to the collar (not pictured).

Referring to FIG. 11, there is provided a perspective top view of a cloak inner layer 1100. In accordance with one embodiment of the present disclosure, the cloak inner layer 1100 comprises a non-absorbent fabric 1152 having a plurality of drainage holes and a sufficient size and shape for a person of any size and weight. The cloak inner layer may be positioned substantially between the side apertures of a cloak (see FIG. 10) detachably attached to a fluid disposal apparatus (not pictured).

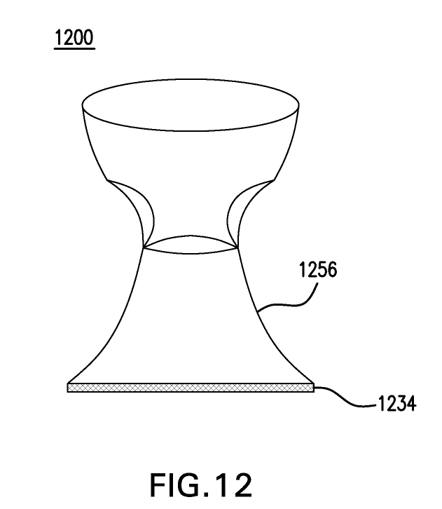

Referring to FIG. 12, there is provided a perspective front view of a cloak 1200. In accordance with yet another embodiment of the current disclosure, a cloak 1200 for providing user privacy is detachably attached to a collar 1234 via a non-absorbent fabric panel 1256 for directing fluid into a fluid disposal system (not pictured).

It should be emphasized that the above-described embodiments of the present disclosure are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A fluid disposal apparatus, comprising:
a liquid collection device;
a perforated support layer disposed above the liquid collection device;
a spacer layer disposed above the perforated support layer comprising a plurality of substantially nonabsorbent members; and
a perforated comfort layer disposed above the spacer layer.

2. The fluid disposal apparatus of claim 1, wherein the liquid collection device comprises a liquid storage container, a funnel disposed adjacent the support layer, and a conduit extending between the funnel and the liquid storage container.

3. The fluid disposal apparatus of claim 1, wherein the liquid collection device comprises one or more cuplike structures affixed to the device.

4. The fluid disposal apparatus of claim 1, wherein any one or more of the support layer, the spacer layer, and the comfort layer comprises a substantially concave drainage channel.

5. The fluid disposal apparatus of claim 1, wherein the perforated support layer is substantially rigid.

6. The fluid disposal apparatus of claim 1, wherein at least one of the perforated support layer and the perforated comfort layer comprises at least one of an open weave structure and a plurality of drainage holes disposed therein.

7. The fluid disposal apparatus of claim 1, further comprising an amount of a substantially firm and substantially flexible cushion disposed on at least one of the plurality of substantially nonabsorbent members of the spacer layer.

8. The fluid disposal apparatus of claim 1, wherein the plurality of substantially nonabsorbent members are finger-like projections.

9. The fluid disposal apparatus of claim 1, further comprising an amount of a substantially firm and substantially flexible cushion disposed on at least a portion of the comfort layer.

10. The fluid disposal apparatus of claim 9, wherein the substantially flexible cushion comprises a flexible gel.

11. The fluid disposal apparatus of claim 1, further comprising a removable top cover for covering at least a portion of the perforated support layer, the spacer layer, and the perforated comfort layer.

12. The fluid disposal apparatus of claim 11, further comprising a collar detachably attached to the removable top cover.

13. The fluid disposal apparatus of claim 12, wherein the collar comprises an outer frame disposed substantially outside an inner frame, the outer frame frictionally detachably attachable to the inner frame.

14. The fluid disposal apparatus of claim 13, wherein the outer frame is attached to the removable top cover.

15. The fluid disposal apparatus of claim 1, wherein the liquid collection device, the perforated support layer, the spacer layer, and the perforated comfort layer are disposed within a bed and removable from the bed.

16. A fluid disposal apparatus, comprising:
a liquid collection device;
a substantially rigid perforated support layer disposed above the liquid collection device, having an open weave structure;
a spacer layer disposed above the support layer, comprising a plurality of substantially rigid, substantially nonabsorbent members, each member comprising an amount of a substantially firm and substantially flexible cushion disposed thereupon; and
a perforated comfort layer disposed above the spacer layer and comprising an amount of a substantially firm and substantially flexible cushion disposed on at least a portion thereof.

17. The fluid disposal apparatus of claim 16, wherein the liquid collection device further comprises a liquid storage container, a funnel disposed adjacent the support layer, and a conduit extending between the funnel and the liquid storage container.

18. The fluid disposal apparatus of claim 16, wherein the liquid collection device comprises one or more cuplike recesses.

19. The fluid disposal apparatus of claim 16, wherein any one or more of the support layer, the spacer layer, and the comfort layer comprises a substantially concave drainage channel.

20. The fluid disposal apparatus of claim 16, wherein at least one of the support layer and the comfort layer comprises a plurality of drainage holes disposed therein.

21. A fluid disposal system, comprising:
a supporting frame;
a fluid disposal apparatus, comprising:
a liquid collection device;
a perforated support layer disposed above the liquid collection device;
a spacer layer comprising a plurality of waterproof members and disposed above the perforated support layer; and
a perforated comfort layer disposed above the spacer layer; and
a padding layer supported by the supporting frame.

22. The fluid disposal system of claim 21, wherein the liquid collection device further comprises a liquid storage container, a funnel disposed adjacent the support layer, and a conduit extending between the funnel and the liquid storage container.

23. The fluid disposal system of claim 21, wherein the liquid collection device comprises one or more cuplike structures affixed to the device.

24. The fluid disposal system of claim 21, wherein any one or more of the support layer, the spacer layer, and the comfort layer further comprises a concave drainage channel.

25. The fluid disposal system of claim 21, wherein the perforated support layer is substantially rigid.

26. The fluid disposal system of claim 21, wherein at least one of the perforated support layer and the perforated comfort layer comprises at least one of an open weave structure and a plurality of drainage holes disposed therein.

27. The fluid disposal system of claim 21, further comprising an amount of a substantially firm and substantially flexible cushion disposed on at least one of the plurality of substantially nonabsorbent members of the spacer layer.

28. The fluid disposal system of claim 21, wherein the fluid disposal apparatus is removable.

29. The fluid disposal system of claim 21, further comprising:
a collar attached to the perimeter of the comfort layer; and
a fabric device cover detachably attached to the collar and disposed adjacent to the comfort layer.

30. The fluid disposal system of claim 29, wherein the fabric device covers is detachably attached to the collar via a draw string.

31. The fluid disposal system of claim 29, wherein the fabric device cover comprises at least one of a padded sheet, a removable and washable absorbent pad, a removable and disposable absorbent pad, a foam pad, a blanket, a rubber mat, and a disposable cotton cushion.

32. The fluid disposal system of claim 29, further comprising:
a cloak for providing user privacy detachably attached to the collar.

33. The fluid disposal system of claim 32, wherein the cloak comprises:
a non-absorbent fabric having a plurality of drainage holes and a sufficient size and shape to surround a person;
a top aperture for accepting the waist of the person;
two lower apertures for accepting the legs of the person; and
a fastening device for securing the cloak to the collar.

34. The fluid disposal system of claim 32, wherein the cloak is attached to the collar via a non-absorbent fabric panel for directing fluid into the system.

35. The fluid disposal system of claim 32, the cloak further comprising a plurality of holes for allowing liquid to flow through to the system.
